(19) United States
(12) Patent Application Publication
(10) Pub. No.: US 2003/0097669 A1
(43) Pub. Date: May 22, 2003

(54) Erk-5 deficient animals and methods of inhibiting angiogenesis through the inhibition of Erk-5

(76) Inventors: Keisuke Kuida, Lexington, MA (US); Michael Shin-San Su, Newton, MA (US); Li Wei, Evanston, IL (US)

Correspondence Address: Andrew S. Marks, VERTEX PHARMACEUTICALS INC., 130 Waverly Street, Cambridge, MA, 02139-4242, US

(21) Appl. No.: 09/922,584

(22) Filed: Aug. 3, 2001

Related U.S. Application Data
(63) Continuation-in-part of application No. 09/888,182, filed on Jun. 22, 2001.
(60) Provisional application No. 60/214,044, filed on Jun. 23, 2000.

(51) Int. Cl. A01K 67/027; C12N 5/06

(52) U.S. Cl. 800/18; 800/8; 435/354

(57) ABSTRACT

The invention relates to transgenic non-human animals, embryos and isolated cells therefrom that are heterozygous or homozygous for a mutation in the extracellular signal regulated kinase, Erk5, gene. Such animals, embryos and cells express Erk5 at a reduced level or not at all. Analysis of the homozygous embryos demonstrates a lack of vasculature, indicating that Erk-5 plays a role in angiogenesis. Thus, the present invention also relates to methods for temporarily decreasing or eliminating angiogenesis in a mammal by administering an agent which inhibits Erk-5 expression or Erk-5 activity. And the present invention also relates to methods for increasing angiogenesis in a mammal by administering a functional Erk5 gene.

### CROSS REFERENCE TO RELATED APPLICATIONS

This application is a Continuation-in-Part and therefore claims priority benefit of co-pending U.S. application Ser. No. 09/888,182 filed Jun. 22, 2001 which claims priority to U.S. Provisional Application Serial No. 60/214,044, filed Jun. 23, 2000. The disclosure of the above priority applications are hereby incorporated by reference.

### TECHNICAL FIELD OF THE INVENTION

The invention relates to transgenic, non-human animals and embryos and isolated cells therefrom that are heterozygous or homozygous for a mutation in the extracellular signal regulated kinase, Erk5, gene. Such animals, embryos and cells express Erk5 at a reduced level or not at all. Analysis of the homozygous embryos demonstrates a lack of vasculature, indicating that Erk5 plays a role in angiogenesis. Thus, the present invention also relates to methods for temporarily decreasing or eliminating angiogenesis in a patient by administering an agent which inhibits Erk5 expression or Erk5 activity. The present invention also relates to methods for increasing angiogenesis in a patient by administering a molecule that increases functional Erk5 expression.

### BACKGROUND OF THE INVENTION

The mitogen-activating protein (MAP) cascades regulate intracellular signaling pathways that are activated by various extracellular stimuli. Three separate MAP kinase cascades have been studied hextensively in mammalian cells—Erk, JNK and p38. In each cascade, three sequentially activated kinases make up the core of the MAP kinase module—a MAP kinase kinase kinase, a MAP kinase kinase and a MAP kinase [M. H. Cobb, Prog. Biophys. Mol. Biol., 71, pp. 479-500 (1999)].

The Erk5 protein represents the newest member of the mammalian MAP kinase family. The human Erk5 consists of 815 amino acids and is nearly twice the size of all known MAP kinases [U.S. Pat. Nos. 5,459,036 and 6,030,822; PCT publication WO 94/21781; J. D. Lee et al., Biochem. Biophys. Res. Commun., 213, pp. 715-24 (1995); and G. Zhou et al., J. Biol. Chem., 270, pp. 12665-69 (1995)].

Erk5 is strongly activated by stresses such as oxidation and hyperosmolarity. Activation by those stimuli is mediated by the MAP kinase kinase kinase, MEKK3, and the MAP kinase kinase, MEK5 [T. H. Chao et al., J. Biol. Chem., 274, pp. 36035-38 (1999)]. Erk5 has been implicated in a signaling pathway initiated by epidermal growth factor [Y. Kato et al., Nature, 395, pp. 713-16 (1998); J. Abe et al., J. Biol. Chem., 271, pp. 16586-90 (1996); Y. Kato et al., EMBO J., 16, pp. 7054-66 (1997)]. Erk5 is also known to phosphorylate myocyte enhancer fact 2c (“MEF2c”) and appears to play a role in neural apoptosis through a p38-dependent mechanism [Z. Mao et al., Science, 286, pp. 785-90 (1999)]. It has also been suggested that Erk5 plays a role in diabetes mellitus, skeletal muscle disease, Alzheimer's disease and peripheral neuropathies [WO 94/21781]. MEKK3 has been implicated in early embryonic cardiovascular development through, but only through its regulation of the kinase p38 [J. Yang et al., Nature Genetics, 24, pp. 309-313 (2000)].

The best way to study the true role of Erk5 in a living organism is to create an organism which does not express Erk5. This can be accomplished using knock-out technology whereby the normal Erk5 gene is mutated in vivo via homologous recombination with a non-functional Erk5 gene introduced into an early stage embryonic stem cell. Heretofore such Erk5 knockouts have not been created. Therefore, there is a need for the creation and analysis of Erk5 knockouts.

### SUMMARY OF THE INVENTION

The present invention solves the problem set forth above by providing non-human animals and embryos that are deficient in Erk5 expression. The invention also relates to cells isolated from those Erk5-deficient animals and embryos.

The embryos of this invention that are homozygous for a mutation in Erk5 in their genome do not survive to birth. Most die at about E9.5 (9.5 days after embryo implantation) and show general delay in development and surprising and unexpected abnormality in the development of yolk sac vasculature. These Erk5 deficient embryos also appeared to lack vasculature in the embryo itself, indicating that Erk5 plays a role in angiogenesis. Thus the present invention provides methods for inhibiting angiogenesis in a patient by inhibiting Erk5 at the DNA, RNA or protein level. This may be achieved by administering to a patient an anti-sense nucleotide targeted specifically for the Erk5 gene or Erk5 mRNA, or tailor-made enzymatic nucleotides akin to ribozymes that specifically cleave Erk5 mRNA. Inhibition at the protein level can be achieved by administering a monoclonal or polyclonal antibody specific for Erk5 or a compound that inhibits the activity of Erk5, typically through competitive binding to the Erk5 active site or a catalytically important auxiliary binding site.

The inhibition of angiogenesis is useful in the treatment and prevention of cancer, hyperplasia, vascular diseases, autoimmune diseases, and certain ocular conditions.

The invention also relates to methods for increasing angiogenesis in a patient by administering to said patient a nucleotide sequence that causes an increase in the expression of functional Erk5 protein via gene therapy techniques. Increased angiogenesis is useful in the treatment of diabetic neuropathic ulcers, other ulcers, limb ischemia, stroke, bone fracture, dementia, head injury or trauma, alopecia, burns and periodontosis, in wound healing, atherosclerosis and in heart bypass surgery to increase collateral blood vessel formation.

### BRIEF DESCRIPTION OF THE DRAWINGS

FIG. 1 depicts the vector pVK/Neo/TK.

FIG. 2 depicts the cloning strategy for creating a non-functional Erk5 gene.

FIG. 3, panels A and B depict the yolk sacs from Embryonic Day (E) 9.5 Erk5 wild-type and mutant mouse embryos, respectively. Panels C and D depict the PECAM stained yolk sacs from an Erk5 wild-type and mutant mouse embryo, respectively. Note that the arrows point to blood vessels. Panels E and F depict a histological, H&E stained section from an Erk5 wild-type and mutant mouse embryo yolk sac, respectively. Note that arrows point to the blood islands.

FIG. 4, panels A and B depict an E10.5 Erk5 wild-type and mutant mouse embryo, respectively. Panels C and D depict the head region of E9.5 embryos, stained with PECAM to reveal vasculature. Panels E and F depict a histological, H&E stained section from an Erk5 wild-type and mutant mouse placenta, respectively. Note that the arrows point to embryonic blood vessels and stars point to maternal blood vessels.

FIG. 5, panels A and B depict a histological, H&E stained section from an Erk5 wild-type and mutant mouse embryo, respectively; as used herein the term “v” refers to the ventricle and the term “a” refers to the atrium. Panels C and D show sections of Erk5 wild-type and mutant mouse embryos stained with Desmin to reveal the myocardium structure.

FIG. 6, panels A and B are Northern blot analysis of erk5 gene expression on staged embryos and adult tissues. Panels C and D show the Control β-actin mRNA level in each blot. Panel pairs E, F and G, H are the Bright-field and dark-field images of E9.5 embryo placenta sagittal sections with Erk5 anti-sense probe, respectively. As used herein, the term “a-vc” refers to the atrio-ventricular canal, the term “ch” refers to the chorion, the term “cv” refers to the common cardinal vein, the term “ec” refers to the ectoplacental cone, the term “fb” refers to the forebrain, the term “hb” refers to the hindbrain, the term “mb” refers to the midbrain, and the term “mc” refers to the maternal cells.

### DETAILED DESCRIPTION OF THE INVENTION

According to one embodiment, the present invention provides transgenic, non-human mammals and embryos and isolated cells therefrom that are heterozygous or homozygous for a mutation in the Erk5 gene. Preferably, the transgenic animals and embryos are mice.

In order to create a non-human animal cell heterozygous for a mutation in the Erk5 gene, one must first provide an Erk5 genomic clone from the same species. This may be achieved by probing isolated genomic DNA or a genomic library from the desired species with an Erk5 specific probe. Such probes can be Erk5-specific primers that, when utilized in conjunction with well-known PCR technology, amplify Erk5-specific genomic DNA. Alternatively, the probe may be an Erk5 cDNA or fragment thereof. Such a cDNA may also be obtained by probing a cDNA library with Erk5-specific primers, followed by PCR. Alternatively, the cDNA may be made by specifically reverse transcribing mRNA encoding Erk5 through the use of Erk5-specific primers. Erk5-specific primers are set forth in the examples below and can also be designed based upon the known cDNA sequence of human Erk5, as set forth in PCT publication WO 94/21781, the disclosure of which is herein incorporated by reference.

Once the Erk5 genomic clone is isolated, the DNA must be mutated so as to render it incapable of encoding a functional Erk5 protein. This may be achieved by a variety of methods well known in the art, such as site-directed mutagenesis or excision of part of the coding region of the gene, with or without concomitant replacement of the excised DNA with alternate DNA.

Preferably, a part of the DNA encoding Erk5 is replaced with DNA encoding a marker gene so that cells transformed with the resulting DNA can be easily identified and selected for. More preferably, a region of the genomic clone encoding Erk5 is replaced by DNA encoding the neo gene, which can later serve as a marker for transformants. Most preferred is when the DNA encoding the non-functional Erk5 also contains a second, different marker gene, such as tk.

The ultimate construct containing the non-functional Erk5 gene is then linearized and used to transform a cell. Preferably, that cell is an embryonic stem cell. The ultimate goal is to have homologous recombination occur between the wild-type Erk5 gene in the chromosomes of the cell and the mutant Erk5 gene in the linearized construct. Cells containing this mutated Erk5 gene are identified by growth in media selective for one or both of the marker genes.

In the case of the neo and tk marker genes, we looked for cells that were resistant to G418 and Gancyclovir®. Homologous recombination is confirmed by Southern blotting against DNA isolated from the transformed cells using a probe specific for the Erk5 gene. The resulting cell, which is heterozygous for a mutation in the Erk5 gene, is one aspect of the present invention.

Once the recombinant cell has been identified and confirmed to have a mutation in the Erk5 gene, it is then injected into a blastocyst from the same species. The resulting chimeric animal is then bred to a normal animal to produce heterozygous offspring. These transgenic offspring, which are heterozygous for a mutation in the Erk5 gene, are another aspect of the present invention.

The chimeric animal may be used as a source of isolated cells which are heterozygous for a functional Erk5 gene. This may be accomplished by taking advantage of the fact that cells which are heterozygous for the Erk5 mutation in such mice are resistant to G418 and Gancyclovir®. Tissues from the chimeric animal are isolated and the individual cells dispersed by well-known techniques. The individual cells are then grown in the presence of G418 and/or Gancyclovir® to select for those which contain the heterozygous Erk5 mutation. Such isolated cells are also part of the present invention and may be used to create cell cultures of cells heterozygous for a mutation in the Erk5 gene.

Individual cells may also be isolated from the heterozygous offspring of the chimera/normal animal cross, either at the embryonic or the post-natal stage, by well-known techniques. Such isolated cells are also part of the present invention and may be used to create cell cultures of cells heterozygous for a mutation in the Erk5 gene. Such cell cultures are useful for studying the effects of reduced expression of Erk5 on cell phenotype and physiology. Those same cell cultures are also useful to assay for compounds that potentially rescue the Erk5 mutation and for evaluating the effects of the gene therapy methods of this invention.

The resulting heterozygous animals are then interbred to obtain an embryo homozygous for a non-functional Erk5 gene. The characterizing feature of animals homozygous for a mutation which causes a functionally deficient Erk5 gene is that they die in the embryonic stage and have a marked reduction or absence of vasculature in the yolk sac and in the embryo itself.

Such embryos and cells isolated therefrom are aspects of the present invention. The cells isolated from the transgenic embryos homozygous for a mutation in the Erk5 gene may be used to create cell cultures which are useful in studying the effects of a lack of expression of Erk5. Such cell cultures are also useful for screening for compounds that are capable of rescuing or compensating for the defect in functional Erk5 expression and for evaluating the effects of the gene therapy methods of this invention.

Confirmation of having produced a genetically altered embryo defective in Erk5 production can be achieved by analysis of that embryo's mRNA or expressed proteins, for the absence of molecules corresponding to Erk5 (mRNA or protein).

Another method of creating cells which are homozygous for a defective Erk5 gene is to perform a second transformation of cells heterozygous for the Erk5 mutation. The second transformation is identical to the first transformation except that a different selection marker is used. Yet another method for creating cells homozygous for a mutant Erk5 gene is to grow the initial transformants in a very high concentration of selectable drug, such that only those cells containing two copies of the marker gene (homozygous for the mutant gene and marker) can proliferate. This method is described in detail in R. M. Mortensen et al., Mol. Cell. Biol., 12, pp. 2391-95 (1992), the disclosure of which is herein incorporated by reference.

According to another embodiment, the invention provides a method of treating or preventing a disease or condition associated with angiogenesis. In particular, the invention provides a method of treating or preventing cancer such as brain cancer, genitourinary tract cancer, lymphatic system cancer, stomach cancer, cancer of the larynx, lung cancer, pancreatic cancer, breast cancer, Kaposils sarcoma, retinoblastoma, neuroblastoma, Wilm's tumor, head and neck cancer, melanoma, colorectal cancer and leukemia; endometriosis, benign prostatic hyperplasia; vascular diseases such as restenosis and atherosclerosis; autoimmune diseases such as rheumatoid arthritis and psoriasis; and ocular conditions such as proliferative or angiogenic retinopathy and macular degeneration.

The above method of this invention comprises the step of administering to a patient suffering from said disease or condition a pharmaceutically acceptable composition comprising a molecule which inhibits either the expression of Erk5 or the activity of Erk5 or a pharmaceutically acceptable salt thereof and a pharmaceutically acceptable carrier. Such molecules include, but are not limited to, monoclonal and polyclonal antibodies specific for Erk5 or epitopes thereof, oligonucleotides that specifically hybridize to Erk5 DNA so as to prevent transcription of functional Erk5 mRNA, oligonucleotides that specifically hybridize to Erk5 mRNA to as to prevent expression of Erk5; ribozyme-like molecules that specifically cleave Erk5 mRNA; and small molecule inhibitors or antagonists of Erk5.

The term “patient” as used herein refers to any mammal, including a human being.

Given that the cDNA sequence and the amino acid sequence of Erk5 are known, as well as methods of utilizing isolated Erk5 to screen for inhibitors, the identification and/or creation of the various Erk5 inhibitors referred to above (at the DNA, mRNA and protein level) is well within the ordinary skill of the art.

For example, the antibodies used in the methods of this invention can be monoclonal or polyclonal or may simply comprise the binding fragment of the antibody or humanized antibodies or fragments. Those antibodies may be raised to intact wild-type Erk5, as well as to fragments of Erk5. Humanized antibodies may be generated using one of the well known procedures in the art, such as chimerization or CDR grafting. Preferably, the antibody or binding fragment thereof will bind to Erk5 and not to any of Erk1, Erk2, Erk3, Erk4 or Erk7.

Techniques for preparing monoclonal antibodies are well known in the art [see, for example, Campbell, “Monoclonal Antibody Technology: Laboratory Techniques in Biochemistry and Molecular Biology”, Elsevier Science Publishers, Amsterdam, The Netherlands (1984); and St. Groh et al., J. Immunol. Methods, 35, pp. 1-21 (1980)]. Spleen cells from the immunized animal are removed and fused with myeloma cells to form monoclonal antibody producing hybridoma cells. The identification of hybridoma cells which produce an antibody with the desired characteristics is achieved through standard techniques such as ELISA, western blot analysis or radioimmunoassay. Selected hybridoma cells are then cloned and the class and subclass of the monoclonal antibody produced thereby is determined using procedures set forth in Campbell, supra.

Techniques for generating polyclonal antibodies are also well known in the art. Any animal (e.g., mouse, rabbit, sheep, etc.) can be immunized with Erk5 or a fragment thereof and will produce antibodies useful in the present invention. Following immunization, antisera is isolated from the immunized animal at various times to determine the presence and titer of Erk5-specific antibodies. Specificity, class and subclass typing are determined as described above.

Oligonucleotides useful in the methods of the present invention may be composed of deoxyribonucleotides, ribonucleotides, DNA/RNA hybrids or a combination of any of the above. Such oligonucleotides will be complementary to sequences present in the Erk5 gene or the mRNA transcribed therefrom. It is preferred that the oligonucleotides used in the methods of this invention have 100% complementarity to a nucleotide sequence present in Erk5. However, oligonucleotide sequences that are less than 100% complementary may also be employed as long as the oligonucleotide binds specifically to Erk5 DNA or mRNA (and does not demonstrate significant non-specific binding to non-Erk5 DNA or mRNA under physiological conditions) and interferes with the normal function of that DNA or mRNA to cause a loss of utility.

The oligonucleotides used in the present invention may target any portion of the Erk5 DNA or mRNA, including, but not limited to, the 5′ untranslated region of the gene or mRNA, the regulatory region of the gene, the promoter region of the gene, the initiation codon of the gene or mRNA, introns present in the gene, any portion of the coding sequence of the gene or mRNA, the termination codon of the gene or mRNA, or the 3′ untranslated region of the gene or mRNA. The oligonucleotides utilized in this invention may be of any length greater than about 8 nucleotides. Preferably, the oligonucleotides are between about 8 to about 30 bases long. Within the scope of the oligonucleotides useful in this invention are those which contain modified bases. Such modifications are well known in the art and are disclosed in WO 00/31296 and references cited therein.

Ribozymes are enzymatic RNA molecules which cleave at specific sites in RNA. Ribozymes specific for the cleavage of Erk5 mRNA may be designed according to well-known methods such as those set forth in WO 93/23569.

Small molecule inhibitors of Erk5 may be identified by any of the methods described in WO 94/21781. Alternatively, one may set up an in vitro kinase assay for Erk5, such as that described in Y. Kato et al., EMBO J., 16, pp. 7054-66 (1997), the disclosure of which is herein incorporated by reference. Such an assay can be used to screen combinatorial or other chemical libraries in an effort to identify compounds that inhibit Erk5 activity.

Pharmaceutically acceptable carriers that may be used in these pharmaceutical compositions include,.but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxy methylcellulose, polyacrylates, waxes, polyethylene-polyoxypropylene-block polymers, polyethylene glycol, sugars such as lactose, sucrose, mannitol, cyclodextrins and derivatives thereof, and wool fat.

If pharmaceutically acceptable salts of the described compounds are used, those salts are preferably derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

Sterile injectable forms of the compositions utilized in the methods of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol. Also, cyclodextrins and their derivatives, as well as phospholipids may be employed to increase solubility.

The pharmaceutical compositions utilized in the methods of this invention may be orally administered in any orally acceptable dosage form including, but not limited to, capsules, tablets, aqueous suspensions or solutions. In the case of tablets for oral use, carriers which are commonly used include lactose, corn starch, cellulose and cellulose derivatives. Lubricating agents, such as magnesium stearate, are also typically added. For oral administration in a capsule form, useful diluents include lactose, dried corn starch, cellulose and cellulose derivatives. When aqueous suspensions are required for oral use, the active ingredient is combined with emulsifying and suspending agents. If desired, certain sweetening, flavoring or coloring agents may also be added.

Alternatively, the pharmaceutical compositions utilized in the methods of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax, and polyethylene glycols.

The pharmaceutical compositions utilized in the methods of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the ERK5 inhibitors utilized in this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions utilized in the methods of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Depending upon the particular condition, or disease, to be treated or prevented, one or more additional therapeutic agents, which are normally administered as a monotherapy to treat or prevent that condition or disease, may be administered to the patient receiving the ERK5 inhibitory pharmaceutical compositions utilized in the methods of this invention. The additional agent may be administered separately, as part of a multiple dosage regimen, from the ERK5 inhibitor-containing composition. In multiple dosage forms, the additional agent may be administered simultaneously with the ERK5 inhibitor or at some period prior to or following the administration of the ERK5 inhibitor-containing composition. Alternatively, the additional agent may be part of a single dosage form, mixed together with the ERK5 inhibitor in a single composition.

For example, chemotherapeutic agents or other anti-proliferative agents may be administered along with the ERK5 inhibitor to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

Other examples of agents that may be administered along with the ERK5 inhibito include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

According to another embodiment, the invention provides methods for increasing angiogenesis in a patient in need thereof. Preferably, the patient is in need of increased angiogenesis due to underexpression of or mutation in its Erk5 gene, decreased native Erk5 mRNA stability, decreased native Erk5 protein stability and/or decreased native Erk5 protein activity. Such methods comprise the step of administering to said patient a chemical entity which causes increased expression of a functional Erk5 protein. Such chemical entities include DNA sequences which comprise an Erk5 coding sequence, DNA sequences encoding a promoter that can be targeted to the Erk5 gene, DNA sequences which allow specific targeting and increased expression of Erk5 (such as those described in U.S. Pat. No. 6,063,630, the disclosure of which is herein incorporated by reference); and homodimeric and heterodimerizing synthetic ligands that allow the ligand-dependent association and disassociation of a transcriptional activation domain with the Erk5 promoter to increase the expression of Erk5 (such as those described in S. N. Ho et al., Nature, 382, pp. 822-26 (1996), the disclosure of which is herein incorporated by reference.

The successful administration of a chemical entity that increases the expression of a functional Erk5 protein to a patient may be achieved by using any of the well-known gene therapy techniques and delivery systems. The methods described above are useful for the treatment of diabetic neuropathic ulcers; wounds; other ulcers, such as those of the skin and digestive organs; limb ischemia, such as fibromuscular dysplasia, thromboangitis obliterans (Buerger's disease), vasculitis, acute arterial occlusion, atheroembolism, Raynaud's phenomenon or Raynaud's disease; stroke; bone fracture; periodontosis; dementia; head injury or trauma; alopecia; burns; atherosclerosis; as well as during heart bypass surgery to increase collateral blood vessel formation.

Preferably, the gene therapy methods are used to treat diabetic neuropathic ulcers; wounds; other ulcers, such as those of the skin and digestive organs; Raynaud's phenomenon, Raynaud's disease and alopecia.

In order that this invention be more fully understood, the following examples are set forth. These examples are for the purpose of illustration only and are not to be construed as limiting the scope of the invention in any way.

### EXAMPLE 1

### Generation of Erk5 Deficient Mice

A pair of Erk5-specific primers: 5′-CAGCCATTCGATGTGGGCCCACGCTA-3′ (SEQ ID NO:1) and 5′-TATAACATTCTCATGGCGGAATCGC-3′ (SEQ ID NO:2) were used to probe isolated mouse genomic DNA.

Using the above primers, a 1.4-kb PCR product was cloned. Partial 5′ (SEQ ID NO:3) and 3′ (SEQ ID NO:4) sequencing of the product confirmed the presence of the expected cDNA segments (exon 2 and exon 3) separated by a 1.2-kb intron (intron 2) [nucleotides 1-38 Of SEQ ID NO:3; and nucleotides 547-794 of SEQ ID NO:4].

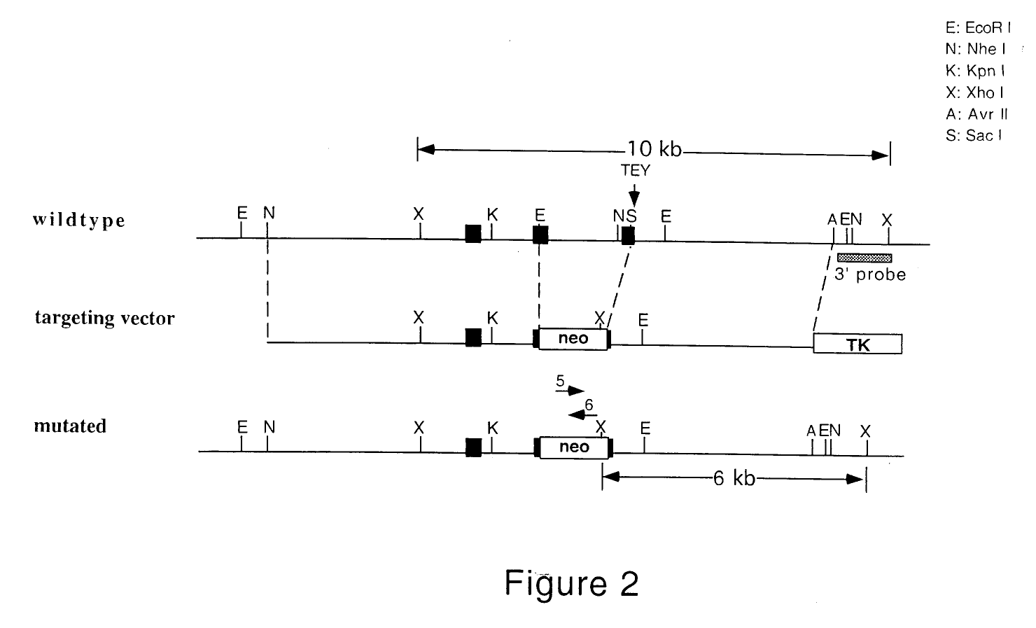

The resulting PCR product was radiolabeled and used to screen a mouse genomic DNA library (strain 129/SV) in phage □□ (obtained from Stratagene, La Jolla, Calif.). This procedure yielded a clone containing a 16-kb Erk5 insert. The phage clone was then subjected to restriction enzyme analysis. The resultant restriction map and subsequent sequencing analysis revealed that a 4.6-kb NheI-EcoRI and a 4.5-kb SacI-AvrII fragment contained a part of exon 2 and a part of exon 3, respectively (FIG. 2).

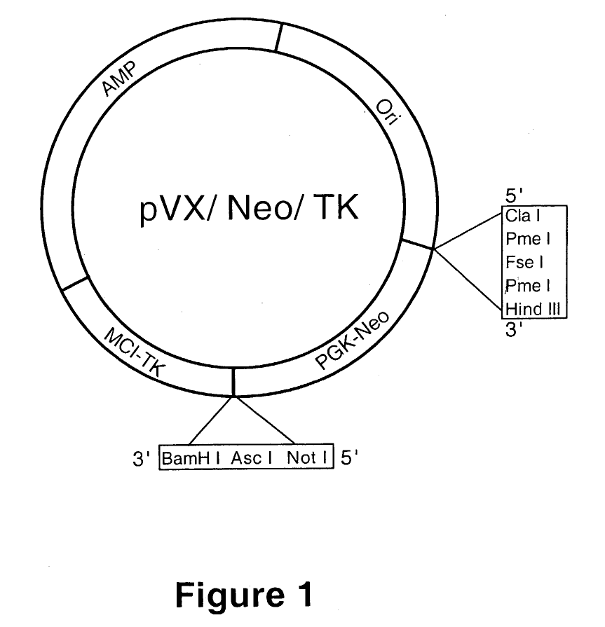

These two fragments were then purified from the phage clone and ligated to FseI and AscI linkers, respectively. The 4.6-kb NheI-EcoRI fragment was subcloned into FseI site of a vector pUX/Neo/Tk (FIG. 1; SEQ ID NO:6); a vector containing neo and tk genes. The 4.5-kb SacI-AvrII fragment was inserted into the AcsI site of the same vector to complete construction of a gene targeting vector for Erk5 gene (FIG. 2). Homologous recombination between the target gene and the vector was expected to result in the deletion of part of exon 2 and exon 3, as well as the deletion of the whole intron 2 of the Erk5 gene.

TC1 embryonic stem (“ES”) cells (obtained from Philip Leder at Harvard University) were transfected with 25 □g of the linearized targeting vector by electroporation and subjected to drug selection with G418 (Gibco, Rockville, Md.) and Gancyclovir®. Genomic DNA from ES cell clones resistant to both of drugs was isolated by conventional DNA isolation technique and digested with XhoI. After gel electrophoresis, DNA was transferred to a nylon filter that was then subjected to Southern hybridization with a radiolabeled 2 kb XbaI-XhoI fragment isolated from the 3′ end of the genomic phage clone (FIG. 2; SEQ ID NO:5 (partial sequence)).

As indicated in FIG. 2, either a 10 kb wild-type band or a 6 kb mutant band would be detected by the probe. Among 250 colonies, five were found by Southern analysis to have undergone the desired homologous recombination event, and two of these (#125 and #145 clones) were injected into C57BL/6 blastocysts to generate two mouse lines.

### EXAMPLE 2

### Genotypic Analysis of Erk-5 Knockout Mice

Mice heterozygous for the mutation were phenotypically normal. Interbreeding of Erk5 heterozygous mice did not generate any homozygous mouse survived to be born (Table 1).

TABLE 1
Genotypes of adult mice generated from
heterozygous breeding.

| Genotype/ | | | | |
|---|---|---|---|---|
| Clone Source | −/− | +/− | +/+ | Total |
| #125 | 0 | 116 | 50 | 166 |
| #145 | 0 | 56 | 36 | 92 |
| Total | 0 | 172 | 86 | 258 |

Further analysis revealed that homozygous mutant embryos began to die at E9.5 and were completely absorbed by E12.5 (Table 2). Homozygous embryos including extra-embryonic tissues looked pale at those stages.

TABLE 2
Embryonic life span of mice with various Erk5 genotypes

| Genotype/ | Embryonic | | | | | |
|---|---|---|---|---|---|---|
| Clone | Age | | | | | |
| Source | (days) | +/− | +/+ | −/− | Absorbed | Total |
| #125 | 8.5 | 15 | 8 | 3 | 0 | 26 |
| | 9.5 | 39 | 14 | 10 | 7 | 70 |
| | 11.5 | 6 | 3 | 1 | 1 | 11 |
| | 13.5 | 5 | 2 | 0 | 2 | 9 |
| #145 | 8.5 | 22 | 10 | 11 | 1 | 44 |
| | 9.5 | 9 | 3 | 3 | 2 | 17 |
| | 11.5 | 10 | 7 | 3 | 4 | 24 |
| Total | | 106 | 47 | 31 | 17 | 201 |

### EXAMPLE 3

### Phenotypic Analysis of Erk5 Knockout Mice

A. Effects on Embryonic Yolk Sac

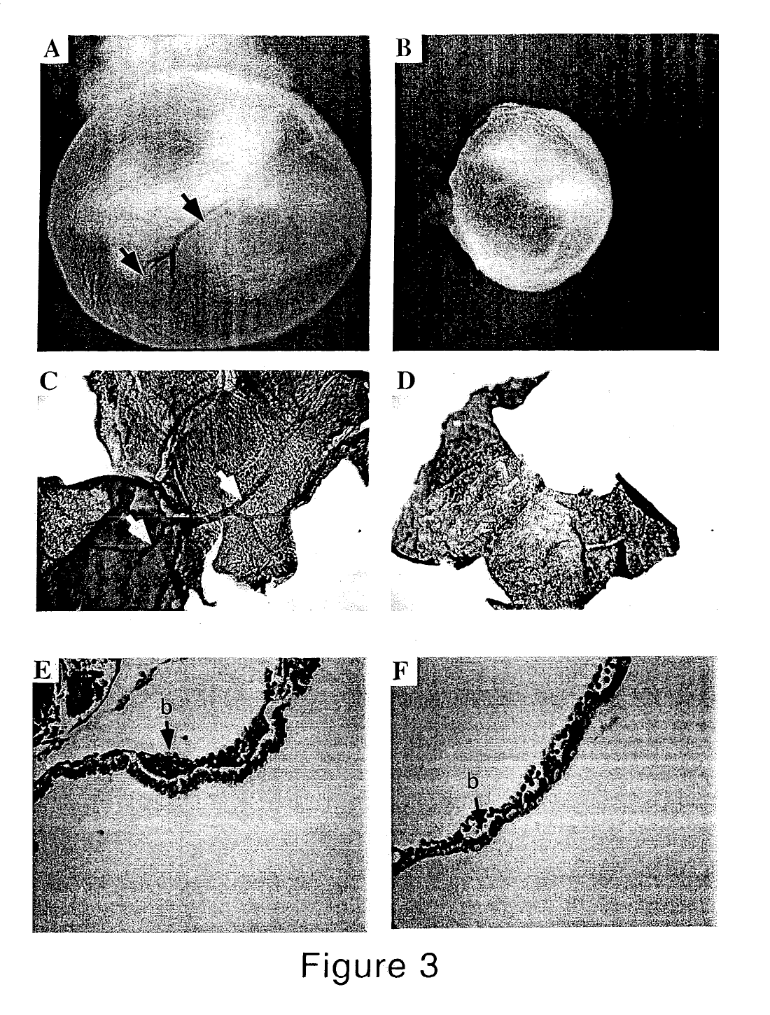

The yolk sac of normal mouse embryos shows early signs of vasculogenesis (FIGS. 3A and 3C). The Erk5 mutant embryo, however, demonstrated a marked lack of yolk sac vasculature at Embryonic Day (E)9.5 (FIGS. 3B and 3D). In addition, blood islands containing hematopoietic cells were recognized in the wild type embryos, but not in the mutant yolk sac by histological analysis (FIGS. 3E and 3F).

B. Effects on Embryonic Development

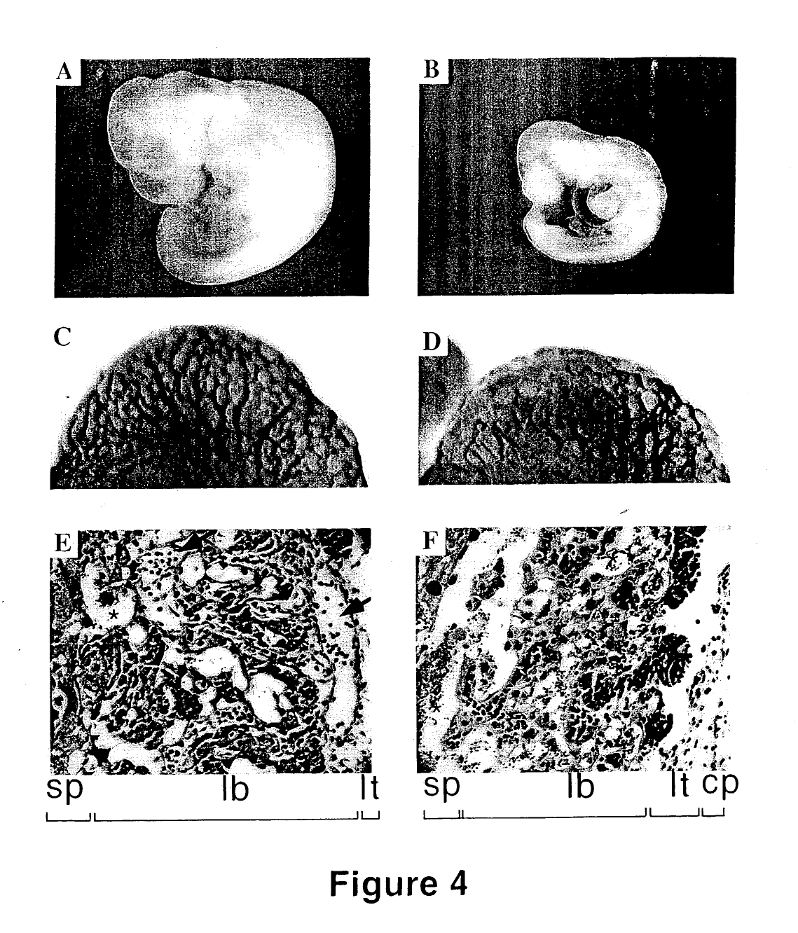

The E10.5 mutant embryos exhibited a general delay in the development due to lack of angiogenesis to support the growth of embryos (FIGS. 4A and 4B).

C. Effects on Placenta and Vascular Complexity

To determine if anomalies in extraembryonic tissues accounted for the embryonic lethality, we examined the placentas of E10.5 embryos. The abnormalities in Erk5 ^{-/-} placentas are shown in FIG. 4F. The mutant labyrinthine region appeared very compact and had very few embryonic blood vessels. Furthermore, there was much less intermingling of maternal and embryonic blood vessels in Erk5 ^{-/-} placenta than that of the wild type. In the wild-type placenta as shown in FIG. 4E, the labyrinthine trophoblasts and vascular endothelial cells were intermingled, whereas in Erk5 ^{-/-} placenta the vascular endothelial cells were mostly restricted to the chorioallantoic region. Vascular endothelial cells were presented in Erk5 ^{-/-} embryos but seemed unable to efficiently invade the labyrinthine region, suggesting a defect in angiogenesis. Vascular complexity, as revealed by staining with an antibody against the endothelial marker(PECAM),particularly in capillaries in the head region, was much simpler in the mutant embryos (FIG. 4D) than that of the wild type (FIG. 4C). Therefore, lack of Erk5 expression affected the angiogenesis process in the development.

D. Effects on the Embryonic Cardiovascular System

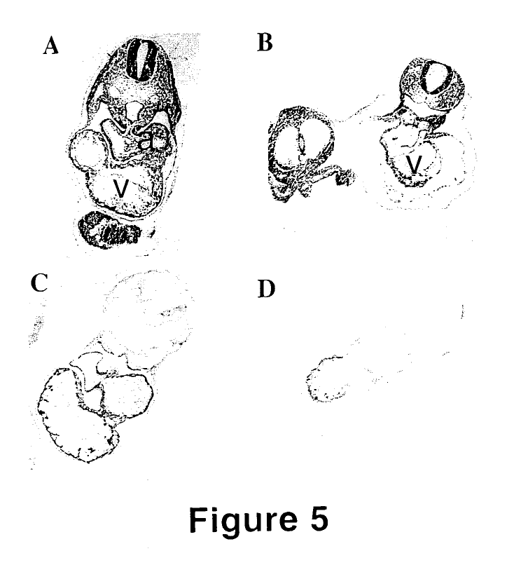

The embryomic cardiovascular system is the first organ system to develop and become critical for fetal survival beyond E9.5 (Srivastava and Olsen, 2000). At the linear heart tube stage (E8.0), the hearts of Erk5 ^{-/-} appeared normal, both phenotypically and histologically (data not shown). At E9.5, however, microscopic analysis revealed that the mutant hearts failed to undergo normal rightward looping. Furthermore, there was pericardial fluid accumulation in the mutant embryos as shown in FIG. 4B, reflecting a failure of normal yolk sac vasculature. These observations were also confirmed by subsequent histological analysis. As shown in FIG. 5A and FIG. B, there was no evidence of the future right ventricle in the mutant hearts while normal hearts clearly had a demarcation for future right and left ventricles. In addition, large space in the pericardium was notable in Erk5 ^{-/-} embryos. At this stage embryonic hearts develop fingerlike protrusions along the inner myocardium, which eventually form the trabeculae. Immunohistological staining of Desmin, the 53 Kda intermediate filament protein abundant in muscle cells, revealed the well-organized trabeculae in the wild-type hearts (FIG. 5C). In contrast, the wall of the mutant hearts was thinner than normal and trabeculation was less obvious (FIG. 5D). These results indicated an essential role of Erk5 in the early heart development.

### EXAMPLE 4

### Expression of Erk5 in Embryonic and Adult Tissues

The expression of Erk5 has been studied primarily on cell lines (Abe, et al., 1996). The expression pattern of Erk5 in the developing fetal and adult tissues has not been reported. Therefore, we performed northern blot analysis both on mouse embryos and on adult tissues and in situ hybridization on embryos.

A. Method

Mouse Multiple Tissue and Embryo Multiple Tissue Northern Blot membranes were purchased from Clontech. An EST clone (W98507) was used to PCR amplify a 530 bp fragment of Erk5 corresponding to amnio acid sequence #678 to #855, with T7 and T3 promoter sequences added onto the 5′ and 3′ of the fragment, respectively. Both strands of Erk5 cDNA clone were separately transcribed by the use of the T7 and T3 phage RNA polymerases, and the product noncomplementary to the mRNA sequence was used as the probe in the negative control. The hybridization was conducted following the instruction by the manufactory. The same membranes were stripped off the Erk5 probes, and re-hybridized with β-actin probe provided by the manufactory.

Detection of Erk5 messages by in situ hybridization was carried out according to Simmons et al. (1989), using ³⁵S-U-labeled RNA probes prepared by the use of a T3/T7 in vitro transcription kit (Ambion). The sources of Erk5 cDNA clones for the preparation of the RNA probe was the same as the one used for Northern Blot analysis. Hybridaization was overnight at 65° C. as described.

B. Results

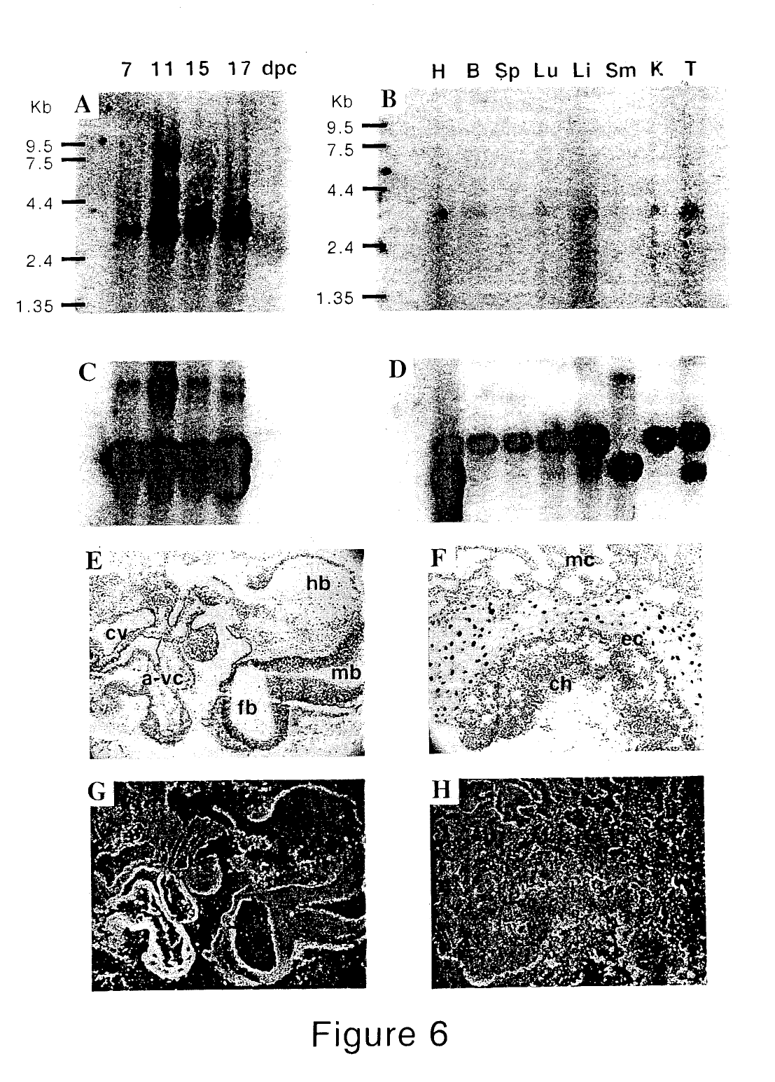

Northern blot analysis of various mouse adult tissues using radiolabeled Erk5 probe showed a ubiquitous presence of a 3.2 kb single transcript (FIG. 6B). While the transcript was seen in adult tissues at a low level, the expression of erk5 was high at various developmental stages (FIG. 6A). After initial probing with anti-sense Erk5 RNA, the blots were washed and rehybridized with β-actin to show uniformity of RNA loading (FIGS. 1C and 1D).

Specificity of the anti-sense Erk5 RNA probe was confirmed by hybridization of both blots with sense Erk5 RNA which did not show any detectable signals (data not shown). In E9.5 embryos, in situ hybridization with the anti-sense Erk5 RNA probe revealed that Erk5 transcripts were expressed at the highest levels in the heart and mesenchyme adjacent to the developing vasculature. The vascular expression of Erk5 was especially apparent in the walls of the common cardinal vein, developing branchial arches and cardiac outflow tract (FIG. 6G). The expression of Erk5 was also apparent in extraembryonic tissues at this stage, noticeably in the placenta. The highest expression was seen in the chorion and diploid trophoblasts in the ectoplacental cone (ec). There was also expression in trophoblast giant cells surrounding ec region (FIG. 6H). These data revealed that expression of erk5 was mainly restricted to the cardiovascular system in the development, suggesting its important role in the vascular development.

The role of Erk5 in angiogenesis is both surprising and unexpected. Previous descriptions of Erk5 suggested that this protein plays a role in stress and is activated by epidermal growth factor (EGF). It has never been suggested that Erk5 was responsible in any way for endothelial cell development or angiogenesis.

While we have described a number of embodiments of this invention, it is apparent that our basic constructions may be altered to provide other embodiments which utilize the products, processes and methods of this invention. Therefore, it will be appreciated that the scope of this invention is to be defined by the appended claims, rather than by the specific embodiments which have been presented by way of example.

We claim:

1. A transgenic non-human mammal whose genome is heterozygous for a mutation engineered into the Erk5 gene, wherein in a homozygous state said mutation results in a functionally deficient Erk5 gene and embryonic death characterized by a lack of vasculogenesis and angiogenesis in said homozygous embryo.

2. A cell isolated from the transgenic non-human mammal according to claim 1, wherein said cell is isolated from said mammal at the embryonic stage or at the post partum stage.

3. A transgenic non-human mammalian embryo whose genome is homozygous for a mutation engineered into the Erk5 gene, wherein said mutation results in a functionally deficient Erk5 gene and embryonic death characterized by a lack of vasculogenesis and angiogenesis in said homozygous embryo.

4. A cell isolated from the transgenic non-human mammalian embryo according to claim 3.

5. An isolated cell heterozygous for a mutation engineered into the Erk5 gene, wherein said mutation results in a functionally deficient Erk5 gene, wherein said cell is produced by introducing a mutated Erk5 gene into a cell containing a functional Erk5 gene.

6. A chimeric non-human mammal which comprises cells that are heterozygous for a mutation engineered into the Erk5 gene, wherein, in a homozygous state, said mutation results in a functionally deficient Erk5 gene and wherein a mammalian embryo whose genome is homozygous for said mutation is characterized by a lack of vasculogenesis and angiogenesis and a failure to survive to birth.

7. A cell isolated from the chimeric non-human mammal according to claim 6, wherein said cell is heterozygous for a defect engineered into the Erk5 gene.

8. The transgenic mammal according to claim 1, wherein said mammal is a mouse.

9. The transgenic mammalian embryo according to claim 3, wherein said embryo is a mouse embryo.

10. The chimeric mammal according to claim 6, wherein said mammal is a mouse.

11. The isolated cell according to any one of claims 2, 4, 5, or 7, wherein said cell is a mouse cell.

12. The isolated cell according to claim 11, wherein said cell is an embryonic stem cell.

13. A method of treating or preventing a condition characterized by angiogenesis in an animal comprising the step of administering to said animal a pharmaceutically acceptable composition comprising a molecule which inhibits any of the transcription of an Erk5 gene, the translation of an Erk5 mRNA or the activity of an Erk5 protein; and a pharmaceutically acceptable carrier.

14. The method according to claim 13, wherein said molecule is selected a monoclonal or polyclonal antibody specific for Erk5, an oligonucleotide that specifically hybridizes to Erk5 DNA so as to prevent transcription of functional Erk5 mRNA, an oligonucleotide that specifically hybridizes to Erk5 mRNA to as to prevent expression of Erk5, a ribozyme that specifically cleaves Erk5 mRNA, or a small molecule inhibitor or antagonist of Erk5 protein.

15. The method according to claim 13, wherein said condition is selected from brain cancer, genitourinary tract cancer, lymphatic system cancer, stomach cancer, cancer of the larynx, lung cancer, pancreatic cancer, breast cancer, Kaposi's sarcoma, retinoblastoma, neuroblastoma, Wilm's tumor, head and neck cancer, melanoma, colo-rectal cancer, leukemia, endometriosis, benign prostatic hyperplasia, restenosis, atherosclerosis, rheumatoid arthritis, psoriasis, proliferative retinopathy, angiogenic retinopathy or macular degeneration.

16. A method for treating a patient in need of increased angiogenesis comprising the step of introducing into said patient a chemical entity which causes increased expression of a functional Erk5 protein in said patient.

17. The method according to claim 16, wherein said patient is characterized by reduced Erk5 activity.

18. The method according to claim 16 or 17, wherein said patient is suffering from diabetic neuropathic ulcers; wounds; other ulcers, such as those of the skin and digestive organs; limb ischemia, such as fibromuscular dysplasia, thromboangitis obliterans (Buerger's disease), vasculitis, acute arterial occlusion, atheroembolism, Raynaud's phenomenon or Raynaud's disease; stroke; bone fracture; periodontosis; dimentia; head injury or trauma; alopecia; burns; or atherosclerosis; or is undergoing heart bypass surgery.

19. The method according to claim 18, wherein said patient is suffering from diabetic neuropathic ulcers; wounds; other ulcers, such as those of the skin and digestive organs; Raynaud's phenomenon, Raynaud's disease or alopecia.

20. The method according to any one of claims 13 to 15, further comprising the step of administering to said patient an additional therapeutic agent which is normally administered as a monotherapy to treat or prevent said condition.

21. The method according to any one of claims 13 to 15, wherein said pharmaceutically acceptable composition further comprises a therapeutic agent which is normally administered as a monotherapy to treat or prevent said condition.


SEQUENCE LISTING (6 sequences)

SEQ ID NO: 1 - DNA, 26 bp, Mus musculus
cagccattcgatgtgggcccacgcta

SEQ ID NO: 2 - DNA, 25 bp, Mus musculus
tataacattctcatggcggaatcgc

SEQ ID NO: 3 - DNA, 802 bp, Mus musculus
  misc_difference (1)..(802): Wherein n is selected from any nucleic acid
cggnacctactgtgccctatggaggaattcagatctgtgtaagggagtgggccaggaggaggagacacagtcgggatcagcttagaagcccaggttcagtaatactgaagttctggcagggcggttgaacccagagtgatgcgggctgtgagtccaggacattggtagggacagttcttatctctcaagagggcaagggctggggatgtcgatcactggtaggctgatgagcatctttgaggttttaggttgactctcctgtacaaaaggggaaaagaatcaagaggatttacctctttatggtcatgccacctttggttatatcataagttcaaggctagtctagaccctgttccaaaagacaaaacanaaaaccnaaacagcaatntanganaagggagagagggcncagacngnccgggacagatccaaattgtaagacaacggacacaatacattgtagtgtcacacagcagtgtcctcatggcagacaactaattattcacagaatacctccttaaaaatagagtcttcaacatagctttttcagtagctgttggcaaactgtagagtttgctctaaaattaaccatactggccaatcttggtagatttgaatatttctataaaaaaaattttttttgacagaaattangtccatggagaaagtgatttgtcagaaagcttgtaaaaaagtttggggctnggaaaaaacccgattcggtgattaagatcactcgatcttttaaaagggacttggctttaantnccataatggnctttcaccggggggcntaaacttt

SEQ ID NO: 4 - DNA, 794 bp, Mus musculus
  misc_difference (1)..(794): Wherein n is selected from any nucleic acid
gattnaagatcccctcgatntttnaaaaggacttggnttcaagggaanagngtnttnncgggggnnaacttgaattgggacnccggtgttgggatcanactccctctttnngcctctgtanacccagggcacccaagtagtacacatacgttcaggaaancatacacatacgtttaagaaaactttataaaagttgtggccagncggtggtggcgcatgcctttaatcccagcactggggaggcagaggcaggcagatctcttgagttttgggtttgaggccaacctggtctacaagagcaagcaagttccaggccagataaggctacacagacatcttgtcttgaaaaaaagaaagaaagaatgaaagttgtagaaaacctaaaacccggtgnnnaantccncncttcccatgntgttagtcctttggggtttcactgtaaggccataacctcaggaattgggagtgccaggggacggagtgccagggggggcttctccctgtgatgtgaggaggctagctcacccgtttcttcccattttcagctatgtggtactggacctcatggagagcgacctacaccagatcattcactcttcacagccgctcaccctggaacatgtgagatacttcctgtaccagctgcttcggggcctcaaatacatgcactctgctcaggtcatccaccgtgatcttaaaccctctaaccttctggtcaatgagaactgtgagctcaagatcggtgactttggaatggcccgtggcctctgtacttccccctgccgagcaccaga

SEQ ID NO: 5 - DNA, 632 bp, Mus musculus
  misc_difference (1)..(632): Wherein n is selected from any nucleic acid
caatcacgggtntnagntcaggntcaagcatggtgccaatgntgagaggcnactccatggcacagttccacttgagcccagggatggcatctcaacatctggacacacaggctcactagccacaggctgcananaagntggaacgnattgttgncgaatgccctccngtcgtgcatgaaagtcttcattctcagccacaatggcctccttaatgcgctccctggtaagggcttcacgggtcaaaagcaaagtcaaaaggtggggcgcaatcaggctcatcatcagggtcatggtacttagccagaaggggtgcgaaggcagcagcagtnagattcgggcnctgggttcaantcgcaccatgcgtcccagcagggagagggcctggcggtcagcacctgggtatactgtctcccaaggcacaggttgccttggtggcaggctctggatataggctcgcaccctttcagcccccacagcctgaatcacagctggtgacggagttcccaacaccatcatgatcagctgtaactggtgcacgtagtttttgcctgggaagagctggcgccgagccagcatctcaccaaagatgcagcccacagaccagaggtcgattgcctgcggtatactcgtgc

SEQ ID NO: 6 - DNA, 617 bp, Mus musculus
  misc_difference (1)..(617): Wherein n is selected from any nucleic acid
ggcaggtaccgcgttagnaccnnttatcngaacccnntgtttntcncagnnnagcnntatttaaccttgnaaanagtttttccctgaggccaagatagcanatangctcnnnggagnncnaaaaaagttttgttctaagaccanngaatnggcagaatgaagtggngaangattagggagantctggaatgacctnantatggtgagtaggaagggaagaaggatcagttaatncagtcacaancnnntgctaactaacgngcctcctntttatgtaagcnattagcancngtttcnngaggcagttggaaattaaaatnttgatatatgttacacacagggccntgcaccacagtagggacttnatgnnntntgggntccagaagagcagtgctgaagggacctgcagctaacttgaaggtactctctggtatatgcccttttcctgctccccaggccagcaggtggccatcaagaagatacctaatgcttttgatgtggtgaccaatgccaaacggaccctcagggagctgaagatcctcaaacacttcaaacacgacaatatcatcgccatcaaggacatcctgaagcctactgtgccctatggagaattcttc